(19) United States
(12) Patent Application Publication
(10) Pub. No.: US 2025/0230267 A1
(43) Pub. Date: Jul. 17, 2025

(54) MICROGEL COMPOSITIONS AND THEIR USE AS LUBRICANTS

(71) Applicant: CORNELL UNIVERSITY, Ithaca, NY (US)

(72) Inventors: David PUTNAM, Ithaca, NY (US); Ruben TRUJILLO, Ithaca, NY (US)

(21) Appl. No.: 18/698,988

(22) PCT Filed: Oct. 6, 2022
(86) PCT No.: PCT/US2022/045843

§ 371 Date: Apr. 5, 2024

Related U.S. Application Data
(60) Provisional application No. 63/252,954, filed on Oct. 6, 2021.

(51) Int. Cl. C08F 120/06; A61K 47/32; A61P 19/02

(52) U.S. Cl. CPC C08F 120/06; A61K 47/32; A61P 19/02; C08F 2438/03

(57) ABSTRACT

A microgel composition comprising micron-sized particles of poly(acrylic acid) in which at least a portion of carboxylic acid groups in the poly(acrylic acid) are crosslinked as shown in the following structure: wherein: L is a linear or branched polyalkylene glycol-containing linker, or L more particularly has the following formula: —O—[CH(R)CH(R′)—O]ₓ—; R and R′ are independently selected from H and CH₃; and x is an integer of 1-50; wherein the micron-sized particles have an average diameter of at least 1 micron. Methods for imparting lubricity to a biological tissue and methods for treating an articular disease or condition (e.g., arthritis or osteoarthritis) by administering the microgel composition are also described.

### CROSS-REFERENCE TO RELATED APPLICATIONS

This application claims the benefit of U.S. Provisional Application No. 63/252,954, filed Oct. 6, 2021, the contents of which is incorporated herein by reference in its entirety.

### STATEMENT REGARDING FEDERALLY SPONSORED RESEARCH

This invention was made with government support under Grant No. DGE-2139899 awarded by the National Science Foundation. The government has certain rights in the invention.

### FIELD OF THE INVENTION

This invention generally relates to pharmaceutically acceptable lubricating compositions and their use in methods of lubricating biological tissue, especially joint, cartilage, and bone surfaces. The invention more particularly relates to methods of using such compositions for treating a variety of conditions, such as osteoarthritis, where lubrication is especially beneficial in treating and ameliorating the effects of the disease or condition.

### BACKGROUND OF THE INVENTION

Osteoarthritis (OA) affects a large segment of the population worldwide. It is associated with cartilage degradation, joint pain, inflammation, altered synovial fluid content, and negatively affects quality of life. Treatment for OA depends on the severity of the disease and includes weight loss, analgesics, non-steroidal anti-inflammatoires (NSAIDs), intraarticular injections of corticosteroids or viscosupplements, and total joint replacement. However, with the exception of total joint replacement, these treatment methods only provide short-term pain relief and do not prevent progression of the disease.

Intraarticular (IA) injection of medications are desirable for OA treatment because they are localized in the joint space with minimal systemic effects. For example, corticosteroids reduce inflammation associated with OA, but their effect is short-term due to rapid clearance from the joint. Within the joint, synovial fluid, comprised of hyaluronic acid, lubricin, and phospholipids, acts as a lubricant and shock absorber. Viscosupplementation is a type of OA therapy that is based on the importance of synovial fluid, primarily hyaluronic acid (HA), and aims to restore the native viscoelastic properties of the healthy joint. HA viscosupplements vary in molecular weight, molecular structure (linear versus crosslinked), and concentration, with a general consensus that high molecular weight hyaluronic acid and crosslinked formulations outperform lower molecular weight formulations. The higher molecular weight and crosslinking leads to more viscous solutions that are equivalent to native synovial fluid, and in some cases exceed synovial fluid viscosity. However, the half-life of HA in a healthy joint is about 20 hours while the half-life in an osteoarthritic joint is about 11-12 hours, which may affect the efficacy of intraarticular HA injections (D. J. Hunter, N. Engl. J. Med., 11, 1040-1047, 2015). Consequently, corticosteroids and HA viscosupplements generally require repeated injections. For viscosupplements, increasing the molecular weight can increase joint residence times; however, there is a viscosity upper limit, above which the material is no longer injectable.

Adequate lubrication of articular cartilage by viscosupplementation correlates positively with clinical outcomes (E. D. Bonnevie et al., PLOS One 14, 1-15, 2019). Therefore, molecular architectures that can function like viscosupplements, that are large enough to be retained in the arthritic joint, and that have viscosities below the injectable limitation, have been sought for the design of next-generation viscosupplements. However, viscosupplements having all of these desired properties have remained elusive.

### SUMMARY OF THE INVENTION

The present disclosure is directed to the design, synthesis, and use of specialized microgel compositions which provide substantial lubrication ability. The microgel compositions described herein function in some way as microscopic ball bearings that decrease the friction between adjoining bone or cartilage tissue. The microgel compositions function as viscosupplements, but with sizes amenable to joint retention and with low viscosity. As further discussed later in this disclosure, a three-level two-factor factorial table was designed to analyze the effects of polymer chain molecular weight and crosslinking density on microgel size, rheological properties, and lubrication. Although viscosity profiles of microgel suspensions were similar to PBS, the microgel composition exhibited lubrication of articular cartilage comparable to bovine synovial fluid and on-market viscosupplements.

The microgel composition contains micron-sized particles of poly(acrylic acid) in which at least a portion of carboxylic acid groups in the poly(acrylic acid) are crosslinked as shown in the following structure:

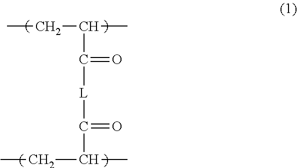

wherein: L is a linear or branched polyalkylene glycol-containing linker, or more particularly, L has the following formula: —O—[CH(R)CH(R′)—O]ₓ—; R and R′ are independently selected from H and CH₃; and x is an integer of 1-50, wherein the micron-sized particles have an average diameter of at least 1 micron. The present disclosure is also directed to pharmaceutical compositions containing the microgel composition and a pharmaceutically acceptable carrier in which the microgel composition is dispersed. The present disclosure is also directed to kits containing the microgel composition and instructions for using the microgel composition.

In another aspect, the present disclosure is directed to methods for imparting lubricity to a biological tissue by contacting the biological tissue with the microgel composition to increase the lubricity of the biological tissue. The biological tissue can be selected from, for example, joints, bone, ocular tissue, nasal tissue, tendons, tendon capsule tissue, intestinal tissue, muscles, and fascia.

In another aspect, the present disclosure is directed to methods for treating an articular disease or condition by administering a pharmaceutically effective amount of the microgel composition to a subject having an articular disease or condition. The articular disease or condition may be, for example, arthritis, such as osteoarthritis.

### BRIEF DESCRIPTION OF THE FIGURES

FIG. 1. Schematics (Parts A and B) showing microgel synthesis: Part A is a chemical schematic of the pAA-TEG microgel synthesis. Part B is a cartoon representation of the pAA-TEG microgel synthesis via a two-phase emulsion to produce spherical microgels.

FIGS. 2A-2D. FIG. 2A shows FTIR qualitative crosslinking density of microgel formulations and pAA-mPEG conjugated polymers. FIG. 2B shows ¹H NMR quantitative crosslinking density of microgel formulations and pAA-mPEG conjugated polymers. Peaks denoted with * represent the dimerization of the acrylic acid monomer. The peak denoted with † represents the methoxy groups found on DMTMM, likely due to residual DMTMM molecules bonded to the carboxylic acids on pAA. FIG. 2C shows representative SEM images of all microgel formulations (scale bar=20 μm). FIG. 2D shows average microgel diameters (n=1236-2733). Statistical significance between groups (p<0.05) is represented by different letters.

FIGS. 3A-3D. Rheological and tribological characterization of microgel suspensions: FIG. 3A is a plot of viscosity profiles of representative microgel suspensions synthesized with pAA_{13.0 kDa} are 1-3 magnitudes less viscous than bovine synovial fluid and Hymovis®. Data points are mean±standard deviation (n=3 for PBS, BSF, and microgel formulations). FIG. 3B is a plot of friction coefficients of PBS, BSF, Hymovis®, and the microgel suspensions at 1 mm/s. Bar graphs are mean±standard deviation (n=6 for PBS, BSF, and microgel formulations, n=4 for Hymovis®). Comparisons between groups were done using a one-way ANOVA and statistical significance between groups (p<0.05) is represented by different letters. Stribeck curves of microgel friction data are shown in FIG. 3C using the measured viscosity values and shown in FIG. 3D using effective viscosity values. Data points are mean±SEM (n=6 per lubricant).

FIGS. 4A-4C. Effects of microgel size, crosslinking density, and concentration on lubrication. FIG. 4A is a plot of average microgel diameter of XLD_{Low} and XLD_{High} microgels. Bar graphs are mean±standard deviation (n=527 and n=553 for XLD_{Low} and XLD_{High}, respectively). FIG. 4B is a plot of friction coefficients of PBS, BSF, XLD_{Low} microgels, and XLD_{High} microgels at 1 mm/s. Bar graphs are mean±standard deviation (n=6 for PBS and BSF, n=3 for XLD_{Low} microgels and XLD_{High} microgels). Comparisons between groups were done using a one-way ANOVA and statistical significance between groups (p<0.05) is represented by different letters. FIG. 4C is a plot of friction coefficients versus sliding speed for XLD_{Low} microgels across various microgel concentrations. Data points are mean±standard deviation (n=6 for PBS and n=3 for XLD_{Low} microgels at all concentrations). Comparisons between groups were done using a two-way ANOVA.

FIGS. 5A-5E. FTIR spectra of microgel formulations synthesized with various crosslinkers demonstrating qualitative crosslinking density: FIG. 5A shows FTIR spectra for pAA_{7 kDa}: PEG600; FIG. 5B shows FTIR spectra for pAA_{7 kDa}: 3-armPEG; FIG. 5C shows FTIR spectra for pAA_{22 kDa}: PEG600; FIG. 5D shows FTIR spectra for pAA_{22 kDa}: 3-armPEG; and FIG. 5E shows FTIR spectra for pAA_{7 kDa}: Cystamine.

FIG. 6. Bar graphs showing friction coefficients of PBS and microgel suspensions synthesized with various crosslinkers at 0.1, 1, and 10 mm/s. Bar graphs are mean±standard deviation (n=1-3 for PBS and microgel formulations); comparisons between groups were done using a two-way ANOVA.

FIG. 7. Bar graphs showing the relative metabolic activity of primary chondrocytes with no treatment or treated with various concentrations of microgels. Bar graphs are mean±standard deviation (n=5 technical replicates, n=3 biological replicates per group); comparisons between groups were done using a two-way ANOVA with a Dunnett's multiple comparisons test).

### DETAILED DESCRIPTION OF THE INVENTION

In a first aspect, the present disclosure is directed to microgel compositions containing micron-sized particles of poly(acrylic acid) (i.e., PAA) in which at least a portion of carboxylic acid groups in the PAA are crosslinked by polyalkylene glycol (PAG)-containing crosslinkers via ester or amide bonds. The “at least a portion” of carboxylic acid groups may be, for example, precisely, about, or at least 3, 4, 4.5, 5, 10, 15, 20, 25, 30, 35, 40, 45, 50, 55, 60, 65, 70, 75, 80, 85, or 90% of the carboxylic groups, or a range bounded by any two of the foregoing values. As well known, the particles in a microgel composition generally form a macromolecular associative network in which each particle is typically swollen with a solvent. For purposes of the present invention, the solvent contained in the microgel should be aqueous-based and non-toxic when administered to a subject. The structure of crosslinked portions of the PAA may be represented by the following structure:

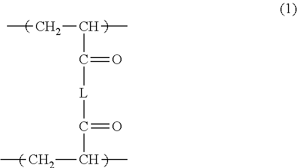

In Formula (1), the variable L is a PAG-containing linker that attaches to at least the two shown carbonyl groups via oxygen atoms (to form ester linkages) or nitrogen atoms (to form amide linkages). In some embodiments, the PAG-containing linker is a polyethylene glycol (PEG)-containing linker or polypropylene glycol (PPG)-containing linker or a linker containing both PEG and PPG units. The PAG-containing linker may be a PAG homopolymer or copolymer. In the case of a copolymer, the copolymer may contain exclusively two or more types of PAG segments, or the copolymer may contain one or more PAG segments and one or more non-PAG segments (e.g., a polyester or polyurethane segment).

In one set of embodiments, the variable L has a linear structure. The linear structure may be represented by the following formula: —O—[CH(R)CH(R′)—O]ₓ—, wherein R and R′ are independently selected from H and CH₃. In some embodiments, R and R′ are both H. In other embodiments, R or R′ is CH₃, or both R and R′ may be CH₃. The variable x is an integer of 1-50. In different embodiments, x is an integer of 1, 2, 3, 4, 5, 6, 7, 8, 9, 10, 12, 15, 20, 25, 30, 35, 40, or 50, or a value within a range bounded by any two of the foregoing values (e.g., 1-40, 1-30, 1-20, 1-15, 1-12, 1-8, 1-6, 2-50, 2-40, 2-30, 2-20, 2-15, 2-12, 2-8, 2-6, 3-50, 3-40, 3-30, 3-20, 3-15, 3-12, 3-8, 3-6, 4-50, 4-40, 4-30, 4-20, 4-15, 4-12, 4-8, 4-6, 5-50, 5-40, 5-30, 5-20, 5-15, 5-12, or 5-8). Any of the values of x or range thereof provided above may be combined with any of the selections of R and R′ also provided above. In some embodiments, a portion of the linear linkers may have only one of its terminal ends attached to a carboxylic acid group while the remaining terminal end of the linker retains its terminal hydroxy group (i.e., dangles) or instead contains a terminal methoxy group.

In another set of embodiments, the variable L has a branched structure. The branched structure contains more than two (typically, three or four) PAG arms emanating from a branching portion to which the arms are attached. The branched linker may be represented by the following formula: W[—O—[CH(R)CH(R′)—O]ₓ—]_{y}, wherein W is a central branched hydrocarbon portion, R, R′, and x are independently as defined above, and y is typically 3 (i.e., three-arm) or 4 (i.e., four-arm). Some examples of three-arm PAG linkers include PAGylated (or more specifically, PEGylated) versions of glycerol, trimethylolethane, trimethylolpropane, triethanolamine, and 1,3,5-trihydroxybenzene (phloroglucinol). Some examples of four-arm PAG linkers include PAGylated (or more specifically, PEGylated) versions of erythritol or pentaerythritol. Notably, although Formula (1) above depicts a linear linker attached to two carboxylic acid groups, Formula (1) is intended to include the possibility of branched linkers, each linking to more than two carboxylic acid groups.

In some embodiments of a three-arm PAG linker, only two of the three PAG arms crosslink between carboxylic acid groups while the remaining PAG arm retains its terminal hydroxy group (i.e., dangles) or instead contains a terminal methoxy group. In other embodiments of a three-arm PAG linker, all three of the PAG arms crosslink between an equivalent number of carboxylic acid groups. Similarly, in some embodiments of a four-arm PAG linker, only two or three of the four PAG arms crosslink between carboxylic acid groups while the remaining two arms or one arm, respectively, retain the terminal hydroxy group or instead contains one or two terminal methoxy groups. In other embodiments of a four-arm PAG linker, all four of the PAG arms crosslink between an equivalent number of carboxylic acid groups.

In some embodiments, the micron-sized particles have a substantially spherical or approximately spherical (e.g., ovoid) and have an average diameter of at least 1 micron. In different embodiments, the micron-sized particles have a diameter of precisely, about, at least, or greater than, for example, 1, 2, 3, 4, 5, 10, 12, 15, 20, 25, 30, 35, 40, 45, 50, 55, 60, 65, 70, 75, 80, 85, 90, 95, or 100 microns, or an average diameter within a range bounded by any of the foregoing values (e.g., 1-100, 1-80, 1-50, 1-40, 1-30, 1-20, 1-10, 2-100, 2-80, 2-50, 2-40, 2-30, 2-20, 2-10, 5-100, 5-80, 5-50, 5-40, 5-30, 5-20, 5-10, 10-100, 10-80, 10-50, 10-40, 10-30, or 10-20 microns). In some embodiments, any of the foregoing diameters or ranges thereof is combined with any of the values of x or ranges thereof and/or selections of R and R′ provided earlier above. In other embodiments, the microgel composition includes particles as described above along with strands of the same PAA crosslinked composition, distributed throughout the microgel composition. Micron-sized particles having any of the above diameters may contain any of the linear or branched crosslinkers (L) described above.

The poly(acrylic acid) (PAA) generally has a molecular weight of at least or above 5 kDa and up to or less than 60 kDa. In different embodiments, the PAA has a molecular weight of precisely, about, at least, greater than, up to, or less than, for example, 5, 6, 7, 8, 9, 10, 12, 15, 20, 22, 25, 30, 35, 40, 45, 50, 55, or 60 kDa, or the PAA has a molecular weight within a range bounded by any two of the foregoing values (e.g., 5-60, 5-55, 5-50, 5-45, 5-40, 5-35, 5-30, 5-25, 5-22, 5-20, 5-15, 5-12, 5-10, 6-60, 6-55, 6-50, 6-45, 6-40, 6-35, 6-30, 6-25, 6-22, 6-20, 6-15, 6-12, 6-10, 7-60, 7-55, 7-50, 7-45, 7-40, 7-35, 7-30, 7-25, 7-22, 7-20, 7-15, 7-12, 7-10, 8-60, 8-55, 8-50, 8-45, 8-40, 8-35, 8-30, 8-25, 8-22, 8-20, 8-15, 8-12, 8-10, 10-60, 10-55, 10-50, 10-45, 10-40, 10-35, 10-30, 10-25, 10-22, 10-20, 10-15, 10-12, 12-60, 12-55, 12-50, 12-45, 12-40, 12-35, 12-30, 12-25, 12-22, 12-20, 12-15, 15-60, 15-55, 15-50, 15-45, 15-40, 15-35, 15-30, 15-25, 15-22, and 15-20 kDa). In some embodiments, any of the foregoing PAA molecular weights or ranges thereof is combined with any of the diameters or ranges thereof provided earlier above and any of the values of x or ranges thereof provided earlier above. Microgel compositions having any of the above PAA molecular weights may contain any of the linear or branched crosslinkers (L) described earlier above.

The PAA is crosslinked with the crosslinker L typically in a crosslinking density of at least 3, 4, or 5%. In different embodiments, the PAA is crosslinked with the crosslinker L in a crosslinking density of precisely, about, or at least 3, 4, 4.5, 5, 10, 15, 20, 25, 30, 35, 40, 45, 50, 55, 60, 65, 70, 75, 80, 85, or 90%, or in a crosslinking density within a range bounded by any two of the foregoing values (e.g., 5-90%, 10-90%, 20-90%, 30-90%, 40-90%, 50-90%, 60-90%, 5-80%, 10-80%, 20-80%, 30-80%, 40-80%, 50-80%, 60-80%, 5-70%, 10-70%, 20-70%, 30-70%, 40-70%, 50-70%, 60-70%, 5-60%, 10-60%, 20-60%, 30-60%, 40-60%, 50-60%, 5-50%, 10-50%, 20-50%, 30-50%, 40-50%, 5-40%, 10-40%, 20-40%, 30-40%, 5-30%, 10-30%, 20-30%, 5-20%, 10-20%, 5-15%, and 5-10%). Notably, the phrase “at least a portion of carboxylic acid groups in the PAA are crosslinked” can be considered equivalent to the crosslinking density, and may be quantified as any of the exemplary crosslinking density values or ranges thereof provided above. In some embodiments, any of the foregoing crosslinking densities is combined with any of the PAA molecular weights or ranges thereof provided earlier above and any of the diameters or ranges thereof provided earlier above and any of the values of x or ranges thereof provided earlier above. Microgel compositions having any of the above crosslinking densities may contain any of the linear or branched crosslinkers (L) described earlier above.

The strands of PAA in the microgel necessarily (i.e., by the laws of chemistry) include a terminating group on each of the two ends of the polymer strand. The terminating group is independently selected from, for example, hydrogen atom, hydrocarbon groups R, or a heteroatom-containing group, such as —OH, —OCH₃, nitrile-containing alkyl (such as provided by the radical initiator or chain transfer agent), thiol (—SH), or dithioester group (as provided by a RAFT chain transfer agent). In some embodiments, at least one of the terminal groups is a thiol group. The terminating groups often correspond to groups originally present in precursor reactants used to synthesize the polymer, and thus, the type of terminating group is often dependent on the chemistry used to synthesize the polymer. Nevertheless, the terminating group may be suitably adjusted by reacting the initially produced polymer to append a specific terminating group, e.g., a cartilage binding domain (e.g., a peptide-containing group) which aids in binding the polymer to a desired biological tissue. In some embodiments, the cartilage binding domain is attached to the polymer via an —S— linker, as in the form RS—, where R is the cartilage binding domain. In some embodiments, the cartilage binding domain is a peptide-containing group (or “peptide”), which may be a monopeptide, dipeptide, tripeptide, or oligopeptide containing at least 4 and up to 5, 6, 7, 8, 9, or 10 peptide units. The cartilage-binding peptide may be, for example, TKKTLRT, SQNPVQP, WYRGRL, SYIRIADTN or CQDSETRFY (SEQ ID. NOs: 1-5, respectively), a cholesterol or other sterol moiety, or any other moiety useful for binding the microgel composition to a biological tissue. Conjugation chemistry for attaching cartilage binding domains, hydrophobic alkyl chains, sterols, or other agents to the polymer are known to those skilled in the art.

In some embodiments, the microgel composition further includes a therapeutic molecule suited for joint therapy or health. Some examples of therapeutic molecules include: (i) non-steroidal anti-inflammatory drugs (e.g., aspirin, ibuprofen, and naproxen), (ii) corticosteroids (e.g., dexamethasone, betamethasone, prednisone, prednisolone, cortisone, hydrocortisone, triamcinolone, and fludrocortisone), (iii) therapeutic proteins (e.g., GBP5, Bcl-2 family pro-apoptotic BH3-only proteins, and sulfatase-2), (iv) protein inhibitors (e.g., tofacitinib, ruxolitinib, and JAK SMIs), and (v) nucleic acids (e.g., mRNA of the transcription factor RUNX1).

In another aspect, the present disclosure is directed to pharmaceutical compositions that contain any of the above-described microgel compositions dispersed in a pharmaceutically acceptable carrier, i.e., vehicle or excipient. The compound may be admixed with, dispersed within, or dissolved in the pharmaceutically acceptable carrier. The microgel composition may be dispersed in the pharmaceutically acceptable carrier by either being mixed (e.g., in solid form with a solid carrier) or dissolved or emulsified in a liquid carrier. The pharmaceutical composition may or may not also contain one or more additional active ingredients or adjuvants that improve the overall efficacy of the microgel composition, particularly as relates to the treatment of articular diseases and conditions.

The microgel composition may be formulated into pharmaceutical compositions and dosage forms according to methods well known in the art. The pharmaceutical compositions of the present invention may be specially formulated for administration in liquid or solid form. The pharmaceutical formulation may be formulated to be suitable for any type of administration, such as oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); intra-articular administration; topical application (e.g., as a cream, ointment, or spray); sublingual or buccal administration; transdermal administration; or nasal administration.

The phrase “pharmaceutically acceptable” refers herein to those substances, materials, compositions, and/or dosage forms which are, within the scope of sound medical judgment, suitable for administration to a subject. The phrase “pharmaceutically acceptable carrier,” as used herein, refers to a pharmaceutically acceptable vehicle, such as a liquid or solid filler, diluent, carrier, manufacturing aid (e.g., lubricant, talc magnesium, calcium or zinc stearate, or stearic acid), solvent, or encapsulating material, that serves to carry the therapeutic composition for administration to the subject. Each carrier should be “acceptable” in the sense of being compatible with the other ingredients of the formulation and physiologically safe to the subject. Any of the carriers known in the art can be suitable herein depending on the mode of administration.

Some examples of materials that can serve as pharmaceutically acceptable carriers, particularly for liquid forms, include water; isotonic saline; pH buffering agents; sugars (e.g., lactose, glucose, sucrose, and oligosaccharides, such as sucrose, trehalose, lactose, or dextran); and antimicrobials. Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

The pharmaceutical composition may also include one or more auxiliary agents, such as stabilizers, surfactants, salts, buffering agents, additives, or a combination thereof, all of which are well known in the pharmaceutical arts. The stabilizer can be, for example, an oligosaccharide (e.g., sucrose, trehalose, lactose, or a dextran), a sugar alcohol (e.g., mannitol), or a combination thereof. The surfactant can be any suitable surfactant including, for example, those containing polyalkylene oxide units (e.g., Tween 20, Tween 80, Pluronic F-68), which are typically included in amounts of from about 0.001% (w/v) to about 10% (w/v). The salt or buffering agent can be any suitable salt or buffering agent, such as, for example, sodium chloride, or sodium or potassium phosphate, respectively. Some examples of additives include, for example, glycerol, benzyl alcohol, and 1,1,1-trichloro-2-methyl-2-propanol (e.g., chloretone or chlorobutanol). If required, the pH of the solutions can be suitably adjusted by inclusion of a pH adjusting agent. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners, and the like may be necessary or desirable. The pharmaceutical formulation may be in the form of a sterile aqueous solution that contains one or more buffers, diluents, and/or other suitable additives such as, but not limited to, penetration enhancers and carrier compounds.

In some embodiments, the carrier further includes a molecular or microscopic (e.g., microscale or nanoscale) sub-carrier in which the microgel composition is loaded, either within and/or conjugated onto the surface of the sub-carrier. The sub-carrier can be composed of, for example, a biocompatible and biodegradable polymer, e.g., based on a polyhydroxyacid biopolyester or polysaccharide. The overall structure of the sub-carrier can be, for example, a micelle, a liposome, dendrimer, nanoparticle, or porous scaffold. These and numerous other types of sub-carriers are well known in the art. The sub-carrier may function to protect the microgel composition during transit, e.g., while in the bloodstream or while passing through the gastrointestinal tract, to release the microgel composition closer to the target cells with lower chance of degradation. The carrier may also function to regulate the rate of release of the microgel composition, such as delayed release or time release. The sub-carrier may also be functionalized with one or more targeting agents that selectively target a class of cells or biological molecules or proteins to be treated with the compound, such as specific receptors in articular tissue.

In embodiments, the microgel composition is administered via an extended release formulation (e.g., sustained release or controlled release) with a release rate, within a predetermined range, of the microgel composition into a biological fluid (or more particularly, articular tissue) of the subject. In embodiments, the extended release within a predetermined range of the microgel composition substantially maintains a minimum predetermined concentration in the biological fluid of the subject within a predefined amount of time. In embodiments, the extended release formulation administered to a subject effects a substantially constant release rate of the microgel composition within the subject over a predefined amount of time. In embodiments, the extended release formulation is a tablet, capsule, microcapsule, micelle, or liposome. In embodiments, extended release is effected via chemical means. In embodiments, extended release is effected via physical means. In embodiments, extended release is effected via both chemical and physical means.

In another aspect, the present disclosure is directed to kits containing any of the microgel compositions described above. The kit typically includes instructions, either as a kit insert with written instructions or with reference to online instructions, for final preparation of and/or use of the microgel composition. The kit may also include one or more devices useful for the administration of the microgel composition (e.g., one or more syringes). The kit may also include one or more reagents useful in preparing a final administrable form of the microgel composition. The kit typically also includes one or more enclosures for holding the microgel composition, instructions, and any devices for its use or preparation.

In another aspect, the present disclosure is directed to methods of synthesizing the microgel compositions. The synthesis of microgel compositions is well known in the art. An exemplary method for synthesizing microgel compositions of the present disclosure entails preparation of a two-phase emulsion in which a PAA and a polyalkylene glycol (PAG) are dissolved in a first solvent (e.g., DMSO) to form a solution. The solution may then be dispersed in a second solvent (e.g., a poloxamer) functioning as a continuous phase, wherein the second solvent may be substantially immiscible with the first solvent or the solvents may be subjected to conditions (e.g., high salt concentration in one of the solvents) that causes one of the solvents to become immiscible in the other solvent. One or more condensing agents (i.e., esterification promoting agents), such as NMM and/or DMTMM, are typically also included in the dispersed phase. The mixture of dispersed and continuous phases is typically emulsified and then homogenized to form a homogeneous dispersion. The dispersed phase is typically in the form of micron-sized droplets in the continuous phase. The microgel can be initially separated from excess liquid phases by centrifugation (typically, at least or above 5000, 6000, 7000, 8000, 9000, 9500, or 10,000 rpm, typically for at least 1, 2, 3, 4, 5, or 10 minutes) to form a pellet from which the liquid material can be decanted. The microgel can then be suspended in a desired liquid phase (typically, non-toxic aqueous phase) to remove remaining solvents used in the preparation of the microgel.

The PAA polymer, before crosslinking, is typically produced by free-radical polymerization. In particular embodiments, the PAA is synthesized by reversible addition-fragmentation chain-transfer (RAFT) polymerization, which is well known in the art. In the RAFT process, a polymer is produced by reacting a vinyl monomer (e.g., acrylic acid) with a RAFT transfer agent (e.g., 4-cyanopentanoic acid dithiobenzoate, i.e., CPA-DB) in the presence of a polymerization initiator (e.g., 4,4′-azobis(4-cyanopentanoic acid), i.e., A-CPA). The reaction conditions typically include heating acrylic acid in solution and in the presence of the RAFT transfer agent and initiator to a temperature of precisely, about, or at least 50, 60, 70, or 80° C. for at least 0.5, 1, 2, 6, 12, 18, 24, 36, or 48 hours or within a range therein.

In another aspect, the invention is directed to methods for imparting lubricity to a biological tissue by using any of the microgel compositions described above. The biological tissue is any biological tissue that could benefit from additional lubricity. The biological tissue may be, for example, joints, bone, ocular tissue, nasal tissue, tendons, tendon capsule tissue, intestinal tissue, muscles, and/or fascia. In the case of bone, the microgel composition can reduce the discomfort, pain, and additional damage resulting from direct bone-on-bone contact, as sometimes occurs in the advanced stages of osteoarthritis. In accordance with this method, biological tissue is contacted with a sufficient (i.e., effective or therapeutically-effective) amount of any of the microgel compositions described herein so as to increase the lubricity or to impart a suitable level of lubricity to the biological tissue. An increased level of lubricity generally corresponds to a lower level of friction (i.e., frictional coefficient, or coefficient of friction, COF) when the biological tissue slides against the same tissue or other material. Frictional coefficients can be measured using a tribometer, which evaluates surface lubrication by linear oscillation of a sample at variable speeds (generally, 0.1, 0.3, 1, 3, and 10 mm/s) and variable compressive normal stresses (generally 250 to 300 kPa). In some embodiments, the method is directed to treating an articular disease or condition by administering a pharmaceutically effective amount of any one of the microgel compositions described above. The articular disease or condition may be, for example, arthritis, or more particularly, osteoarthritis.

As used herein, the terms “sufficient amount,” “therapeutically-effective amount,” and “effective amount” are used interchangeably to refer to an amount of a microgel composition of the invention that is sufficient to result in sufficient lubricity of a biological tissue, or the prevention of the development, recurrence, or onset of the disease or condition (e.g., osteoarthritis) or one or more symptoms thereof, or to enhance or improve the prophylactic effect(s) of another therapy, reduce the severity and duration of the disease or condition, ameliorate one or more symptoms of the disease or condition, prevent the advancement of disease or condition, and/or enhance or improve the therapeutic effect(s) of additional treatment(s).

A therapeutically-effective amount of the microgel composition can be administered to a patient in one or more doses sufficient to palliate, ameliorate, stabilize, reverse, or slow the progression of the disease or condition, or otherwise reduce the pathological consequences of the disease or condition, or reduce the symptoms of the disease or condition. The amelioration or reduction need not be permanent, but may be for a period of time ranging from at least one hour, at least one day, or at least one week, or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form, regimen, and the desired result. In certain embodiments of the invention, the therapeutically effective amount is an amount that is effective to treat the condition (e.g., osteoarthritis), achieves pain relief over a period of time, improves joint movement and flexibility, and/or reduces friction in the joint or other accepted measure of improvement in the treatment. The dosage level may be within a range from 1-50 mg/mL, or more particularly, 1-40 mg/mL, 1-30 mg/mL, or 1-20 mg/mL. In exemplary embodiments, dosage levels range from about 8-22 mg/mL in injection volumes of 2-4 mL (for humans), and more typically about 10 mg/mL in an injection volume of 3 mL.

The biological tissue to be lubricated can be contacted with any of the microgel compositions by means well known in the medical arts. The biological tissue can be contacted with the microgel composition by, for example, injecting, infusing, implanting, spraying, or coating the microgel composition directly into or onto the biological tissue, or indirectly into biological tissue surrounding the tissue to be lubricated. In general, contacting a biological tissue means that the microgel composition is delivered to the tissue in any manner that leads to coating of the surface or bathing of the tissue with the microgel composition. In certain embodiments, the tissue is contacted by injection or infusion of the composition into a joint space, thereby leading to a coating of cartilage and/or the meniscus found in that joint space. Moreover, the volumes used are at least partly dependent on the type of tissue being contacted, whether a space is being filled, or a surface is being coated, as could be determined by one skilled in the medical arts.

In some embodiments, the microgel composition is injected or infused into or onto an arthritic or injured joint or bone to improve the lubricity of the joint or bone. As such, the microgel composition provides boundary lubrication. The treatment may be specifically directed for treating or preventing osteoarthritis. The treatment of osteoarthritis or an injured joint, cartilage, or bone preferably results in reduction of symptoms, improved mobility, less joint pain, and overall inhibition of disease progression, or prophylaxis in the case of an injured joint. The method can also comprise administering one or more of the microgel compositions described above along with simultaneous or sequential administration of another composition that functions to augment or work in tandem with the microgel composition. The augmenting (i.e., auxiliary) composition may be selected from, for example, hyaluronic acid, lubricin, synovial fluid, glycosaminoglycan, or other auxiliary agent. These other agents can also be administered by, for example, injection or infusion. In some embodiments, these other agents may work synergistically with one or more of the microgel compositions described above to provide enhanced lubrication and wear protection.

In particular embodiments, the biological tissue is a joint, cartilage, or bone, and more typically, an injured or arthritic joint, cartilage, or bone. In some embodiments, the joint is a weight bearing joint, such as a hip, knee or ankle joint. Many different joints can benefit from an increased level of lubricity, including the shoulder, elbow, wrist, hand, finger and toe joints. Nevertheless, the biological tissue being lubricated is not limited to joints, cartilage, and bone. Other biological tissues that may be lubricated by use of the disclosed microgel composition include gastrointestinal tissue, eye tissue, nasal tissue, and vaginal tissue. Thus, by use of the microgel composition described herein, a variety of conditions may be treated beyond those associated with joints, cartilage, and bone. Some of these other conditions include, for example, dry eye syndrome, dry nose, carpal tunnel syndrome, post-menopausal vaginal dryness, and more. Those skilled in the medical arts can determine the appropriate delivery route and method for contacting a particular biological tissue. For example, for dry eyes, contacting may be achieved by instilling drops; for dry nose, contacting may be achieved by nasal spray; for carpal tunnel syndrome contacting may be achieved by injecting near or around the inflamed tendon and capsule; and for post-menopausal dry vagina, a pill, troche or suppository can be placed in or implanted in the vagina. Hence, this method can be used to achieve boundary mode lubrication for any of a wide variety of biological tissues that could benefit from additional lubrication.

Examples have been set forth below for the purpose of illustration and to describe the best mode of the invention at the present time. However, the scope of this invention is not to be in any way limited by the examples set forth herein.

### EXAMPLES

### Synthesis and Characterization of Lubricating Microgels

### Poly(Acrylic Acid) Synthesis

Poly(acrylic acid) (pAA) was synthesized by reversible addition-fragmentation chain transfer (RAFT) polymerization, as previously reported (J. L. Rios et al., Pharm. Res. 33, 879-892, 2016; J. M. Pelet et al., Macromol. Chem. Phys. 213, 2536-2540, 2012). A standard polymerization started with purifying the acrylic acid monomer by removing MEHQ inhibitor in an aluminum oxide column. Appropriate amounts of the CTA (CPA-DB) and initiator (A-CPA) were weighed out depending on the desired molecular weight of the product and placed in a round-bottom flask. Methanol (40 mL) and the purified acrylic acid (10 mL, 145.85 mmol) were added to the round-bottom flask and the system was purged with nitrogen gas for 15 minutes to remove excess air. After the removal of air, the round-bottom flask was sealed and then placed in an oil bath at 60° C. After 48 hours, the reaction was quenched in liquid nitrogen. A crude sample was taken for proton nuclear magnetic resonance (¹H NMR, 500 MHz, D20) to determine the conversion of the reaction and the polymer was purified by dialyzing against DI water for three days. Finally, the purified polymer was flash frozen in liquid nitrogen and lyophilized until dry. NMR was performed on the dried polymer to confirm complete removal of residual methanol, chain transfer agent, initiator, and unreacted monomer. Gel permeation chromatography (GPC) was used to analyze the polydispersity, weight molecular average, and number molecular average of the pAA.

### Microgel Synthesis

Microgels were synthesized using a two-phase microemulsion with DMSO as the dispersed phase and Pluronic L35® (a PEG-PPG-PEG triblock poloxamer) as the continuous phase (see J. L. Rios et al. Ibid.). The carboxylic acid groups on the pAA backbone were condensed with hydroxyl groups on TEG to form a hydrogel network. NMM and DMTMM were used as condensing agents to facilitate the esterification. The total amount of NMM for each reaction differed to achieve different crosslinking densities. pAA (120 mg, 1.67 mmol COOH) was dissolved in DMSO (1.5 mL). DMTMM (276.5 mg, 1.00 mmol DMTMM) and varying amounts of NMM were added to the solution and dissolved with stirring (450 rpm) for 1.5 hours. The Pluronic L35® was separated into pre-emulsion (15 g in a glass vial) and homogenization (25 g in a 100 mL beaker) vessels. To the DMSO solution was added TEG (143.8 μL, 1.67 mmol of OH groups) and the solution was stirred at 450 rpm for two minutes. The dispersed phase was then pipetted into the pre-emulsion Pluronic L35® vial and vortexed for three minutes to create a homogenous pre-emulsion. The pre-emulsion was then added to the homogenization Pluronic L35® in the beaker (25 g) and the mixture was homogenized at 750 rpm for four hours at room temperature using a homogenizer with a 1-inch slotted head.

After the reaction was complete, microgels were pelleted by centrifugation at 9500 rpm at 25° C. for five minutes. The supernatant was decanted using a serological pipette and microgels were resuspended by vortex in 25 mL of DI water. DI water (25 mL) was added to the microgels, followed by sonication (10 minutes) using an ultrasonic cleaner. Microgels were left suspended in 50 mL of DI water for an additional 20 minutes unperturbed and at room temperature after the first wash to allow any solvent, Pluronic L35®, and unreacted reagents to diffuse into the water phase. Microgels were subsequently pelleted at 5000 rpm at 25° C. for 5 minutes. The supernatant was decanted and the microgels were resuspended in 3% w/v glycine (10 mL) and incubated overnight at 4° C. to remove any remaining activated carboxyl groups. Notably, the microgels may be washed with molecules other than glycine, including, for example, other amino acids (e.g., alanine, valine, or serine), carboxylic acids (e.g., acetic acid or propanoic acid), dicarboxylic acids (e.g., oxalic acid, malonic acid, or succinic acid), alcohols (e.g., ethanol or isopropanol), diols (e.g., ethylene glycol or propylene glycol), and polyols (e.g., glycerol). Microgels were washed the following day by 3 cycles of re-suspension in 50 mL of deionized water, centrifugation at 5000 rpm at 25° C. for 5 minutes, and isolated by removing the supernatant. Lastly, microgels were suspended in DI water (5 mL), flash frozen in liquid nitrogen, and lyophilized at room temperature to dryness.

Microgels were additionally synthesized using a syringe pump and syringe filter for studies elucidating microgel size dependence and concentration dependence. To do this, the same protocol was followed as described above to form a homogenous pre-emulsion. The pre-emulsion was then added to a 60 mL syringe with a 0.8 μm syringe filter and vertically injected into the homogenization Pluronic L35® using a syringe pump at a rate of 2.5 mL/min. This mixture was then added back to the syringe and filtered an additional two times. After the last filtration, the mixture was left to stir at 250 rpm for 4 hours. Microgels were washed in the same manner as described above.

To determine the effect of microgel size on lubrication, microgels of different sizes were separated by differential size centrifugation. Microgels were centrifuged at 1000 rpm at 25° C. for one minute. The supernatant containing the “small” size fraction of microgels was decanted and placed in a separate centrifuge tube. “Small” size fraction and “large” size fraction microgels were centrifuged once more at 5000 rpm at 25° C. for five minutes. Microgels were suspended in 5 mL of DI water, flash frozen in liquid nitrogen, and lyophilized at room temperature to dryness.

### Microgel Size Analysis

Scanning electron microscopy (SEM) was used to characterize the size and morphology of the microgel formulations. Conductive double-sided carbon tape was placed on SEM pin stub mounts. Microgel formulations for SEM were prepared via the droplet evaporation technique with microgels suspended in deionized water (0.05-0.1 mg/mL). SEM stubs containing microgels were dried in a desiccator, then sputter-coated with gold and palladium. Microgels were imaged at a working distance of 9 mm at 5 kV. Microgel size was determined using a custom MATLAB code.

### Qualitative and Quantitative Crosslinking Density Analysis by Fourier-Transform Infrared Spectroscopy (FTIR) and NMR

Qualitative crosslinking density of microgel formulations was determined using FTIR. Three microgel batches with the same molecular weight of pAA and different crosslinking densities were analyzed. KBr was dried overnight in an oven to remove moisture. FTIR pellets (13 mm diameter) were made with 300 mg KBr and 5 mg microgels and compressed to 10 tons with a die kit. Samples were analyzed from 400 cm⁻¹-4000 cm⁻¹ with a resolution of 4 cm⁻¹ and 64 total scans. Absorbance data were baseline corrected and normalized to the largest peak.

Quantitative crosslinking density was determined by replicating the microgel reaction conditions and substituting methoxy PEG (mPEG, containing only one reactive OH group) in place of the TEG crosslinker. pAA (120 mg, 1.67 mmol COOH) was weighed into a glass vial and dissolved in DMSO (1.5 mL). DMTMM (276.5 mg, 1.0 mmol DMTMM) and varying amounts of NMM (11 μl, 0.1 mmol for low conjugation; 54.9 μL, 0.5 mmol for medium conjugation; 109.9 μL, 1.0 mmol for high conjugation) were added to the solution and stirred at 450 rpm for 1.5 hours at room temperature. After 1.5 hours, mPEG (841 μL, 1.67 mmol OH) was added to the reaction mixture and allowed to stir for 4 hours at 200 rpm. The reaction mixture was purified by dialysis against DI water for at least 3 days and lyophilized to dryness. ¹H NMR was performed on the dried polymer (n=3) to determine the total conjugation percentage of the mPEG on the pAA backbone. Conjugation percentage was calculated by taking the integral of the —CH₃ peak from the mPEG (3.54-3.88 ppm), dividing by 3 to account for the three hydrogens, and finally dividing by the integral of the —CH— backbone from the poly(acrylic acid) (2.22-2.72 ppm).

### Rheology

The viscosity of microgel formulations was measured using a commercial rheometer. Microgels were suspended in PBS (2.5 mg/mL) and tested using a 40 mm aluminum parallel plate geometry with a 500 μm gap width, and a logarithmic shear rate sweep from 1-1000 l/s. All tests were conducted at a temperature of 20° C. and 10 data points were collected per decade (n=3 per lubricant).

### Lubrication Studies

Lubrication induced by microgel suspensions was measured using a custom-built tribometer, as previously described (E. D. Bonnevie et al., PLos One 14, 1-15, 2019; E. Feeny et al., J. Heat Transfer, 142, 1-10, 2020). Femoral condyles from the stifle joint of neonatal bovine were harvested and used to make condyle plugs that measure 6 mm wide by 2 mm thick. The cartilage plugs were incubated for 30 minutes in 1.5 M NaCl in PBS to remove native lubricin from the cartilage surface. The plugs were then incubated in PBS with protease inhibitor for 1 hour to remove any remaining NaCl. The cartilage plugs were then glued to brass pivots and placed in the tribometer wells with the microgels, compressed to 30% strain, and allowed to stress relax for 1 hour until they reach an equilibrium normal load. The glass counterface was articulated via a DC motor and the load cells measured the shear force and normal load during sliding. The tribometer platform slides at predetermined speeds ranging from 0.1 mm/s to 10 mm/s. The friction coefficient was calculated as the ratio of the average shear load while sliding to the average normal load while sliding. All microgel batches were tested at a concentration of 2.5 mg/mL in PBS unless otherwise specified. Bovine synovial fluid (BSF) and PBS were used as a positive and negative controls, respectively (n=6 per lubricant). Friction data were further analyzed by plotting the friction coefficients versus the Sommerfeld number (Eq. 1) for PBS and BSF,

$$S = ( v * {η}_{0} * a ) / {F}_{N} \qquad ( Eq . 1 )$$

where v is the sliding speed, is the zero-shear viscosity of the lubricant, a is the contact width of the cartilage plug, and FN is the normal load of the cartilage plug. A model Stribeck curve was then fit to the PBS and BSF data using Eq. 2 for the friction coefficient as a function of the Sommerfeld number,

$$μ ( S ) = {μ}_{min} + ( {μ}_{B} - {μ}_{min} ) * exp ( - ( S / {S}_{t} ) \hat{} d ) \qquad ( Eq . 2. )$$

where μₘᵢₙ is the minimum friction coefficient, μ_{B} is the boundary friction coefficient, Sₜ is the Sommerfeld transition number, and d is a fitting parameter.

The values obtained for the model Stribeck curve are: μₘᵢₙ=0.046, μ_{B}=0.26, Sₜ=1.81*10⁻⁷, and d=0.31. Microgel friction data was plotted as a function of the respective Sommerfeld numbers using the measured viscosity values at 10 s⁻¹. The effective viscosity was calculated as detailed previously with modifications (E. D. Bonnevie et al. and E. Feeney et al., Ibid.). Briefly, for each microgel batch evaluated, a custom MATLAB code was used to generate 50 random friction curves based on experimental data. The viscosity value in Eq. 1 was allowed to change to calculate a predicted Sommerfeld number. Then, the predicted Sommerfeld number was used in Eq. 2 to calculate a theoretical friction coefficient using the previously determined model parameters. The root-mean-square (RMS) error between the theoretical friction coefficients and the measured friction coefficients were minimized by continuing to vary the viscosity value in Eq. 1. The final viscosity value when the RMS error was minimized was the effective lubricating viscosity.

### In Vitro Cytocompatibility

Chondrocytes were isolated from neonatal bovine condyles using sterile practices. Condyles were cut into cubes (approx. 1 mm3), washed three times with PBS containing 1% antibiotic/antimycotic (ABAM), and incubated with a 0.3% collagenase type II solution for 18 hours. Digested condyles were then pipetted through 100 μm cell strainers into conical tubes and cells were isolated by three washes of centrifugation, decanting of the supernatant, and resuspending in sterile PBS with 1% ABAM. Cells were washed one final time in PBS with ABAM and resuspended in Dulbecco's Modified Eagle Media (DMEM with 4.5 g/L glucose, 10% fetal bovine serum (FBS), and 1% ABAM) at 2.994×10⁶ cells/mL. Cells were plated on a 96-well plate at a density of 5×10⁴ cells/well and incubated at 37° C. with 5% CO₂ for 48 hours before treatment. Prior to cell treatment, microgels were suspended in 10 mL of 70% ethanol and subsequently washed with three cycles of sterile PBS with 1% ABAM. Microgels were finally suspended in an appropriate amount of cell media to achieve the desired individual concentrations. Microgels with varying crosslinking densities were incubated with the chondrocytes at different concentrations (2.5 mg/mL, 1 mg/mL, 0.1 mg/mL, 0.01 mg/mL, and 0.001 mg/mL) to determine the dose-response of the microgels on cell viability. Cells treated with microgels were compared directly to cells without any treatment (n=5 technical replicates per plate, n=3 biological replicates per group). An MTT assay was performed two days post-treatment according to the manufacturer's protocol. Absorbance was read on a microplate reader at 570 nm.

### Statistics

A two-way ANOVA was used to analyze the average diameters of the microgel batches, aggregated at the picture level, with main effects of crosslinking density, pAA molecular weight, and their interaction. The model assumptions of normality and homogeneous variance were assessed visually using residual plots. Post-hoc pairwise comparisons between batches were performed using Tukey's HSD method to control the Type I error rate. A two-way ANOVA was used to analyze the effects of sliding speed and lubricant on the friction coefficients. A one-way ANOVA was used to analyze the effects of the lubricant on the friction coefficients at an individual speed. Dunnet's multiple comparisons test was performed on MTT data to assess the effects of microgel concentration on cell viability relative to the control. Differences between groups were considered significant at p≤0.05 for all statistical tests.

### Results and Discussion

### I. Generation of a Microgel Library

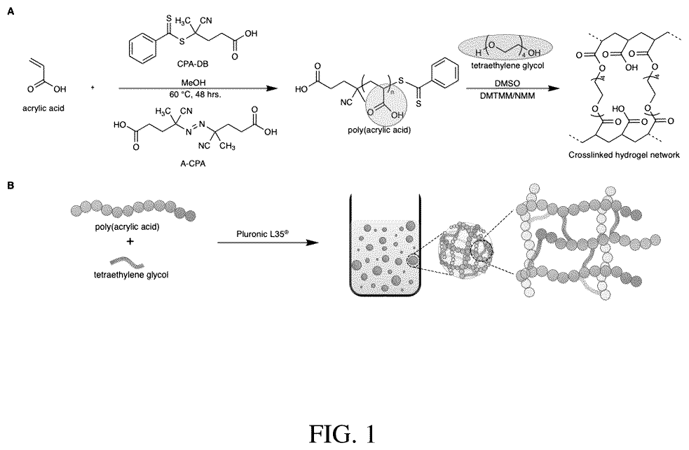

Polymeric microgels, composed of poly(acrylic acid) and tetraethylene glycol (TEG), were synthesized via a two-phase emulsion using DMSO as the dispersed phase and Pluronic L35® as the continuous phase. A schematic of the process is provided in FIG. 1, parts (a) and (b). The choice of pAA and TEG for the microgel synthesis offers many benefits, including biocompatibility, facile post-polymerization conjugations via the carboxylic acid groups on pAA, and post-synthesis cargo loading, which bypasses the relatively harsher reaction conditions. In the microgel synthesis, multiple parameters were identified that could ultimately affect the microgel sizes, viscosity profiles, and lubrication. These parameters include pAA molecular weight, crosslinking density, crosslinker length, type of crosslinker, and homogenizer speed. Ultimately, it was determined that the most important parameters to evaluate were the pAA molecular weight and crosslinking density of the microgels because the viscosity of polymeric solutions varies with polymer molecular weight due to chain entanglement, and changing the crosslinking density of the microgels would directly affect the mesh size, swelling ratio, and the number of free carboxylic acid groups (e.g., N. A. Peppas et al., Eur. J. Pharm. Biopharm. 50, 27-46, 2000). For this study, a two-level three-factor factorial table (Table 1), below, was created which allowed for the complete analysis of the effects of pAA molecular weight and crosslinking density on microgel properties. The pAA molecular weight used for a specific microgel batch is herein denoted as pAA_{X} where ‘X’ represents the respective molecular weight. The pAA crosslinking density (XLD) is denoted as and XLD_{Y} where ‘Y’ represents either low, medium, or high crosslinking density. pAA was successfully synthesized and characterized using RAFT polymerization as previously reported (e.g., J. L. Rios et al., Ibid.). RAFT polymerization allows parity between the theoretical and experimental molecular weights, and low polydispersity (Ð=1.21-1.31), which were confirmed by both ¹H NMR and GPC. Microgels were prepared with three distinct pAA molecular weights: 6.9 kDa (pAA6.9 kDa), 13.0 kDa (pAA13.0 kDa) and 22.7 kDa (pAA22.7 kDa). These pAA molecular weight targets were chosen to remain below the cut-off for renal filtration (30-50 kDa).

Table 1. Three-level two-factor factorial table of microgel formulations varying pAA molecular weight and crosslinking density. The COOH:NMM ratio represents the molar ratio between the carboxylic acid side chains on the pAA and the NMM.

| | | | Crosslinking Density |
|---|---|---|---|
| Microgel Batch | | pAA Mₙ (kDa) | (COOH:NMM) |
| pAA_{6.9 kDa} | XLD_{Low} | 6.88 | 1:0.06 |
| | XLD_{Med} | | 1:0.3 |
| | XLD_{High} | | 1:0.6 |
| pAA_{13.0 kDa} | XLD_{Low} | 13.04 | 1:0.06 |
| | XLD_{Med} | | 1:0.3 |
| | XLD_{High} | | 1:0.6 |
| pAA_{22.7 kDa} | XLD_{Low} | 22.70 | 1:0.06 |
| | XLD_{Med} | | 1:0.3 |
| | XLD_{High} | | 1:0.6 |

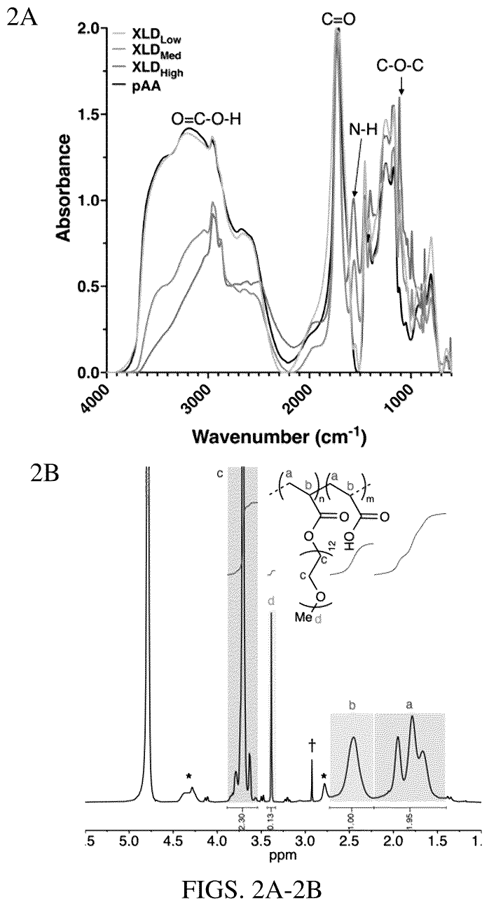

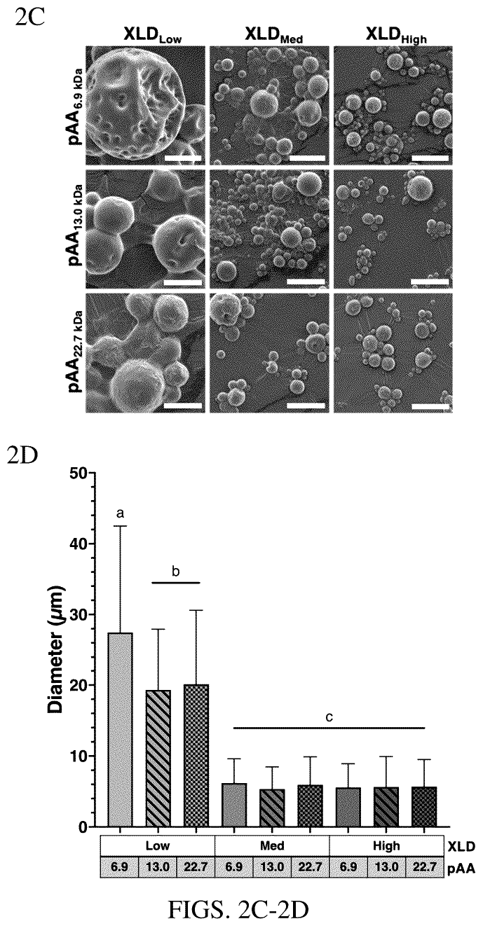

Microgel crosslinking density was verified both qualitatively and quantitatively using FTIR and ¹H NMR for XLD_{Low}, XLD_{Med}, and XLD_{High} microgels. FTIR showed both a decrease in the carboxylic acid peak (O—H stretch between 2500-3500 cm⁻¹) and an increase in the ether peak (C—O—C stretch at 1110 cm⁻¹) relative to pAA, indicating the incorporation of TEG into the microgels via esterification (FIG. 2A). Using ¹H NMR to measure the crosslinking density of microgels through the use of a mPEG conjugation, the percent conjugation of XLD_{Low}, XLD_{Med}, and XLD_{High} microgels was calculated to be 4%, 12%, and 15%, respectively (FIG. 2B). Crosslinking density was varied by changing the amount of NMM added to the reaction solution. The reaction outcome was non-linear with the reaction efficiency showing a plateau for the XLD_{Med} and XLD_{High} microgels, which is consistent with the relevant DMTMM:NMM synthesis literature (e.g., J. L. Rios et al., Ibid.).

To visualize microgel morphology, SEM was used in combination with a custom MATLAB code to determine the average microgel size for each batch. Microgels exhibited spherical morphology and different crosslinking densities led to different sized particles (FIG. 2C). Tukey comparisons showed that the average diameter was significantly greater for batches with XLD_{Low} than those with XLD_{Med} or XLD_{High} (p<0.0001). Additionally, for the XLD_{Low} batches, the average diameter was significantly greater for the pAA6.9 kDa batch compared to the pAA13.0 kDa or pAA22.7 kDa batches (p<0.0001, FIG. 2D). Microgels with XLD_{Low} had an average diameter between 19-28 μm while particles with XLD_{Med} and XLD_{High} had an average diameter between 5-6 μm.

II. Polymer Microgels Achieve High Levels of Lubrication with Low Viscosity

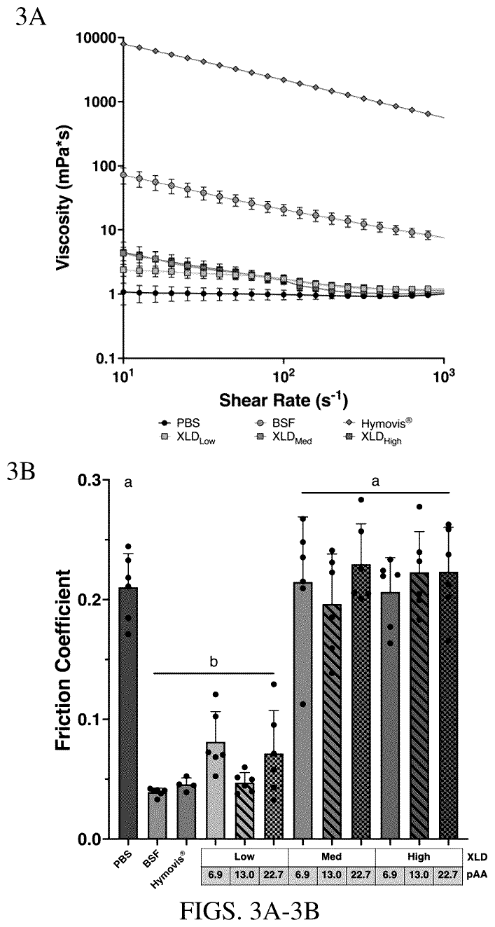

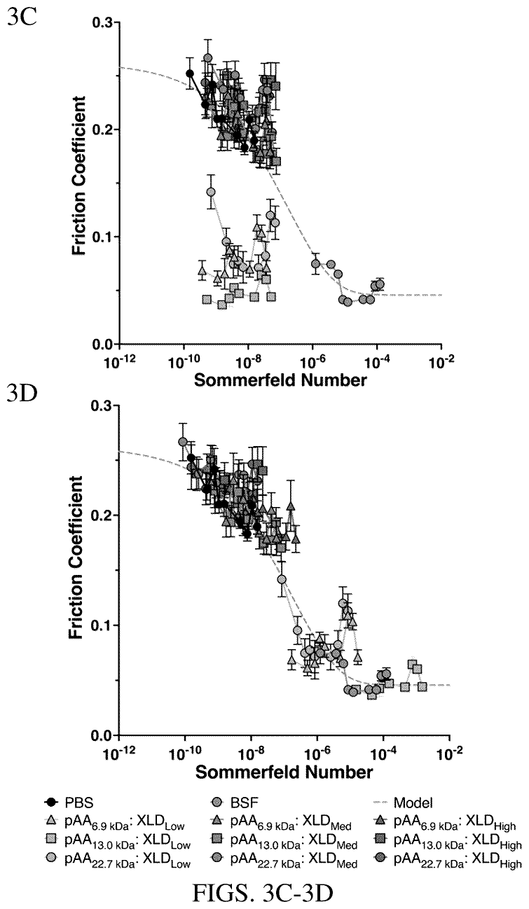

The viscosity of synovial fluid, primarily due to hyaluronic acid (HA), contributes to its lubricating and shock absorbing properties. Within native synovial fluid, hyaluronic acid concentrations vary from 2.5-4 mg/mL, while viscosupplements remain more concentrated to achieve highly viscous solutions (e.g., D. J. Hunter, N. Engl. J. Med., 11, 1040-1047, 2015). Therefore, rheology was performed on microgel suspensions (2.5 mg/mL) to determine their viscosity profiles relative to natural synovial fluid and therapeutic HA viscosupplements. When comparing the viscosity profiles of the microgel suspensions to bovine synovial fluid (BSF) and Hymovis®, an on-market viscosupplement, it was herein observed that the microgel formulations were 1-3 magnitudes less viscous (FIG. 3A). The viscosity data was obtained from a Carreau-Yasuda model curve, as described in E. D. Bonnevie et al., Ibid. Indeed, the microgel suspensions had viscosity profiles comparable to PBS (˜0.001 Pa*s), independent of the crosslinking density and pAA molecular weight. Similar studies of polymer nanospheres, hyaluronic acid microgels, and biodegradable mesoporous silica nanoparticles (MSNs) demonstrated similar results for viscosity profiles, with viscosity profiles only being slightly more viscous than PBS (e.g., K. Zhang et al., Chem.—A Euro. J., 26, 10564-10574, 2020).

Viscosupplementation, as the name suggests, aims to restore the joint's function by providing lubrication through viscosity restoration. Current on-market viscosupplements have zero-shear viscosity values on the order of 0.5-190 Pa*s in an attempt to mimic hyaluronic acid, a primary lubricating component of native synovial fluid. The rheological results for this study also correspond to empirical equations by Einstein, Batchelor, and Krieger and Dougherty that describe the viscosity of particle suspensions as a function of volume fraction, only leading to a large increase in viscosity at high volume fractions.

Tribological characterization of microgel suspensions was performed on a custom-built tribometer platform to determine the friction coefficients as a function of sliding speed. Across all sliding speeds, microgels synthesized with medium molecular weight pAA and low crosslinking density (pAA13.0 kDa: XLD_{Low}) showed no significant differences in friction coefficients when compared to BSF. The results additionally demonstrate that microgel suspensions with low crosslinking density, at all pAA molecular weights, have superior lubrication compared to PBS (p<0.001) and lubricate articular cartilage equivalent to BSF and Hymovis®, while microgel batches with medium and high crosslinking density had friction coefficients equivalent to PBS (FIG. 3B). Decreased friction coefficients relative to PBS and water have been achieved in other nanoparticle and microparticle suspension systems. The various tribological systems that can be used to calculate friction (pin-on-disks, cartilage-on-cartilage, cartilage-on-glass), along with various experimental conditions (applied load, rate/speed of articulation, stress relaxation of cartilage, etc.), make direct comparisons of friction coefficients difficult. However, comparisons can be made relative to positive and negative controls. Specifically, in nanoparticle suspension systems, friction has been decreased by 30-71.2% relative to DI water/PBS. For micron-sized suspension systems, a reduction in friction compared to DI water/PBS has been observed between 29.6-50% (e.g., Y. Han et al., Bioact. Mater., 6, 3596-3607, 2021). Lubrication of articular cartilage has also been observed in modified hyaluronic acid, hyaluronic acid mimetic polymers, and lubricin mimetic polymers, decreasing friction relative to PBS between 60.8-77.5%. Microgel suspensions used in this study successfully decreased the friction coefficient relative to PBS by as much as 83.5%, exhibiting competitive lubrication relative to other systems.

In the field of tribology, Stribeck curves are used as the standard to distinguish various lubrication modes based on the force balance between the lubricant and the sliding substrate. When plotting the microgel friction curves on a Stribeck curve using measured viscosity values, the microgel suspensions did not follow normal Stribeck curve behavior and were clustered in the mixed lubrication regime due to their similar viscosities (FIG. 3C). For these tribometric studies, the contact width, normal load, and sliding speed are consistent across all lubricants, with the viscosity being the only variable that differs across lubricants and would therefore shift the Sommerfeld numbers. However, deviations from classical Stribeck behavior indicate unique lubricant interactions at the cartilage interface, leading to friction curves that are shifted relative to the model Stribeck curve. The discrepancy from classic behavior derives from the realization that conventional rheology measurements conducted with steel surfaces do not accurately reflect viscosity measurements of lubricants conducted at cartilage surfaces. In fact, viscosity values measured between steel surfaces were shown to be much lower when compared to cartilage surfaces (S. G. Cook et al., ACS Biomater. Sci. Eng., 6, 2787-2795, 2020). Additionally, the viscosity of hyaluronic acid solutions between cartilage surfaces increased as gap width decreased, which begins to more accurately mimic conditions in vivo with small gaps between articular cartilage. The shift in Stribeck behavior is corrected by calculating the effective viscosity of each lubricant, which reflects the viscosity of a lubricant directly at the cartilage-glass interface, while under compression.

When effective viscosities are used for the Stribeck curve, large shifts of Sommerfeld numbers are revealed (FIG. 3D). XLD_{Med} and XLD_{High} batches had low effective viscosities and were shifted to the boundary mode lubrication regime. XLD_{Low} batches exhibited effective viscosities 2-4 magnitudes larger than viscosities measured by rheology, resulting in a shift to larger Sommerfeld numbers in the elastoviscous lubrication regime (Table 2, shown below). Effective viscosities correlate to clinical outcomes of viscosupplements and are used to understand the behavior of shear-thinning systems in soft contacts. Large differences between measured and effective viscosities suggest that microgels with low crosslinking density interact with the cartilage surface during sliding, resulting an increased viscosity at the cartilage-glass interface.

Table 2. Measured viscosities and effective viscosities of microgel suspensions. Low XLD batches experienced an increase in effective viscosity while Med XLD and High XLD experienced a decrease in effective viscosity.

| Microgel Batch | | η(10 s⁻¹) (mPa*s) | η_{effective} (mPa*s) |
|---|---|---|---|
| pAA_{6.9 kDa} | XLD_{Low} | 2.04 ± 1.17 | 935 ± 530 |
| | XLD_{Med} | 3.15 ± 0.65 | 3.60 ± 4.70 |
| | XLD_{High} | 3.79 ± 4.49 | 12.4 ± 24.5 |
| pAA_{13.0 kDa} | XLD_{Low} | 2.38 ± 0.50 | 76200 ± 39600 |
| | XLD_{Med} | 4.31 ± 1.02 | 4.70 ± 4.60 |
| | XLD_{High} | 4.44 ± 1.99 | 1.80 ± 1.70 |
| PAA_{22.7 kDa} | XLD_{Low} | 3.52 ± 3.26 | 574 ± 430 |
| | XLD_{Med} | 4.71 ± 2.22 | 0.57 ± 0.55 |
| | XLD_{High} | 3.22 ± 2.97 | 1.10 ± 1.10 |

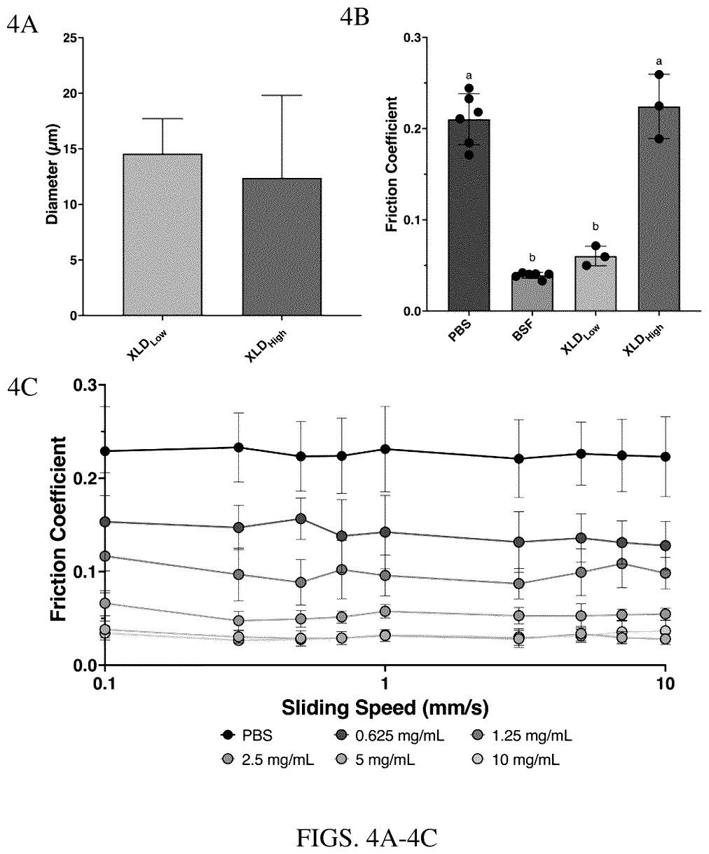

Microgel batches with low crosslinking density successfully lubricated cartilage, but they were also significantly larger than medium and high crosslinking density microgels. To investigate whether lubrication was dictated by microgel size or by crosslinking density, microgels of similar sizes were synthesized with low and high crosslinking density (FIG. 4A). When the lubrication characteristics of similar sized microgels were evaluated, microgels with XLD_{Low} continued to exhibit superior lubrication compared to XLD_{High} (FIG. 4B). These data confirm that microgel suspensions lubricate as a function of crosslinking density, independent of microgel size. For hydrogels, as crosslinking density increases, mesh size decreases, which ultimately affects the degree of swelling. As crosslinking density decreases for the microgel formulations, there are more available carboxylic acid groups to interact with the aqueous environment, and it is possible this contributes to lubrication through a hydration lubrication mechanism. Generally, hydrogel lubrication is thought to result from high solvent swelling and can occur through mechanisms that involve hydrodynamic forces of fluid flow through the hydrogel network, absorption or repulsion between the gel and the opposing substrate, and micromechanical and thermodynamic properties of the hydrogel network (N. L. Cuccia et al., Proc. Natl. Acad. Sci. U.S.A., 117, 11247-11256, 2020; J. P. Gong, Soft Matter, 2, 544-552, 2006). Additionally, hydrogels with larger mesh sizes generally have lower friction coefficients, which support the present findings that microgels with low crosslinking density (larger mesh sizes) have improved lubrication over microgels with medium and high crosslinking density.

Small molecule drugs, proteins, and large molecules, such as hyaluronic acid, have short half-lives within the joint space, which are further decreased by the onset of osteoarthritis (e.g., D. J. Hunter, Ibid.). Micron-sized particles have been shown to have an increased residence time in vivo compared to smaller suspension systems due to their size (e.g., S. J. Cross et al., ACS Biomater. Sci. Eng., 6, 5084-5095, 2020). Independent of residence time, lubrication of microgel suspensions as a function of concentration would help determine proper doses for an optimum microgel treatment to achieve sufficient joint lubrication comparable to synovial fluid. Therefore, the dose dependence of lubrication of articular cartilage as a function of microgel concentration was explored. Microgels with XLD_{Low} and an average diameter of 22.5±3.1 μm were evaluated at concentrations varying from 0.625-10 mg/mL. Lubrication using XLD_{Low} microgels followed a dose dependent response and lubricated equivalent to BSF across all sliding speeds beginning at 2.5 mg/mL while still maintaining low viscosity values (n (10 s⁻¹)=4.82 mPa*s at 10 mg/mL) (FIG. 4C). Ultimately, microgels achieved lubrication equivalent to BSF across 4× dilution and relatively low weight percent (maximum 1 wt/v %) compared to other systems, which points to the effectiveness of a microgel treatment that lasts throughout multiple half-lives.

The objective of this study was to determine the effects of pAA molecular weight and crosslinking density on microgel size, rheological properties, and lubricating abilities using a three-factor two-level factorial table. The results presented herein demonstrate the therapeutic ability of microgel compositions for OA treatment. The present work demonstrates the successful lubrication of articular cartilage with pAA and TEG microgels. Using the factorial table for this study, the effects of pAA molecular weight and crosslinking density on microgel size, viscosity, and lubrication were studied. From the data, it is clear that crosslinking density directly affects microgel size and lubrication. Stribeck curve analysis shows that when classic rheology is used to measure viscosity, these microgel suspensions do not show differences, but using the effective viscosity, the low crosslinking density microgels collapse along the classic Stribeck curve, suggesting that there is an interaction between the microgels and the cartilage surface. Regardless of pAA molecular weight, low crosslinking microgel suspensions exhibited superior lubrication compared to high crosslinking microgel suspensions, and they lubricated articular cartilage equivalent to BSF across 4× dilution.

Microgels were synthesized with various crosslinkers and crosslinking densities, similar to the methods described above. For these syntheses specifically, the Pluronic L35® was separated into pre-emulsion (15 g in a glass vial) and homogenization (25 g in a 100 mL beaker) vessels. To the DMSO solution was added the appropriate crosslinker (PEG600, 3-arm PEG, and cystamine dihydrochloride) and the solution was stirred at 450 rpm for two minutes. The dispersed phase was then pipetted into the pre-emulsion Pluronic L35® vial and vortexed for three minutes to create a homogenous pre-emulsion. The pre-emulsion was then added to the homogenization Pluronic L35® in the beaker (25 g), the mixture was homogenized at 750 rpm for 10 minutes at room temperature using a homogenizer with a 1-inch slotted head, and subsequently left to mix with a stir bar at 250 rpm for 2-4 hours. The molar ratios for each microgel reaction are shown below in Table 3.

Table 3. Summary of microgel batches synthesized with various crosslinkers. The COOH:RLG:DMTMM:NMM ratios represent the molar ratios between the carboxylic acid groups, the reactive linker groups (RLG), DMTMM, and NMM, respectively. The number of reactive linker groups for any linker are based on the functionality of the linker. For example, linear PEG molecules have two reactive linker groups while branched PEG molecules, such as 3-arm PEG, have three reactive linker groups.

| | pAA | | | Reaction |
|---|---|---|---|---|
| | Mₙ | | | Time |
| Batch | (kDa) | Linker Type | COOH:RLG:DMTMM:NMM | (hours) |
| pAA_{7 kDa}: | 7 | Polyethylene | 1:1:0.6:0.06 | 4 |
| PEG600_{Low} | | glycol (Mₙ ≅ | | |
| | | 600 Da) | | |
| pAA_{7 kDa}: | 7 | Polyethylene | 1:1:0.6:0.6 | 4 |
| PEG600_{High} | | glycol (Mₙ ≅ | | |
| | | 600 Da) | | |
| pAA_{7 kDa}: | 7 | 3 arm PEG | 1:1:0.6:0.06 | 4 |
| 3armPEG_{Low} | | (Mₙ ≅ 450 Da) | | |
| pAA_{7 kDa}: | 7 | 3 arm PEG | 1:1:0.6:0.6 | 4 |
| 3armPEG_{High} | | (Mₙ ≅ 450 Da) | | |
| pAA_{22 kDa}: | 22 | Polyethylene | 1:1:0.6:0.06 | 4 |
| PEG600_{Low} | | glycol (Mₙ ≅ | | |
| | | 600 Da) | | |
| pAA_{22 kDa}: | 22 | Polyethylene | 1:1:0.6:0.6 | 4 |
| PEG600_{High} | | glycol (Mₙ ≅ | | |
| | | 600 Da) | | |
| pAA_{22 kDa}: | 22 | 3 arm PEG | 1:1:0.6:0.06 | 4 |
| 3armPEG_{Low} | | (Mₙ ≅ 450 Da) | | |
| pAA_{22 kDa}: | 22 | 3 arm PEG | 1:1:0.6:0.6 | 4 |
| 3armPEG_{High} | | (Mₙ ≅ 450 Da) | | |
| pAA_{7 kDa}: | 7 | Cystamine | 1:1:0.6:0.06 | 4 |
| Cystamine_{Low} | | dihydrochloride | | |
| pAA_{7 kDa}: | 7 | Cystamine | 1:0.4:0.3:0.03 | 2 |
| Cystamine_{High} | | dihydrochloride | | |

FTIR and tribological experiments were performed to determine qualitative crosslinking density and friction coefficients of microgel batches, as described above.

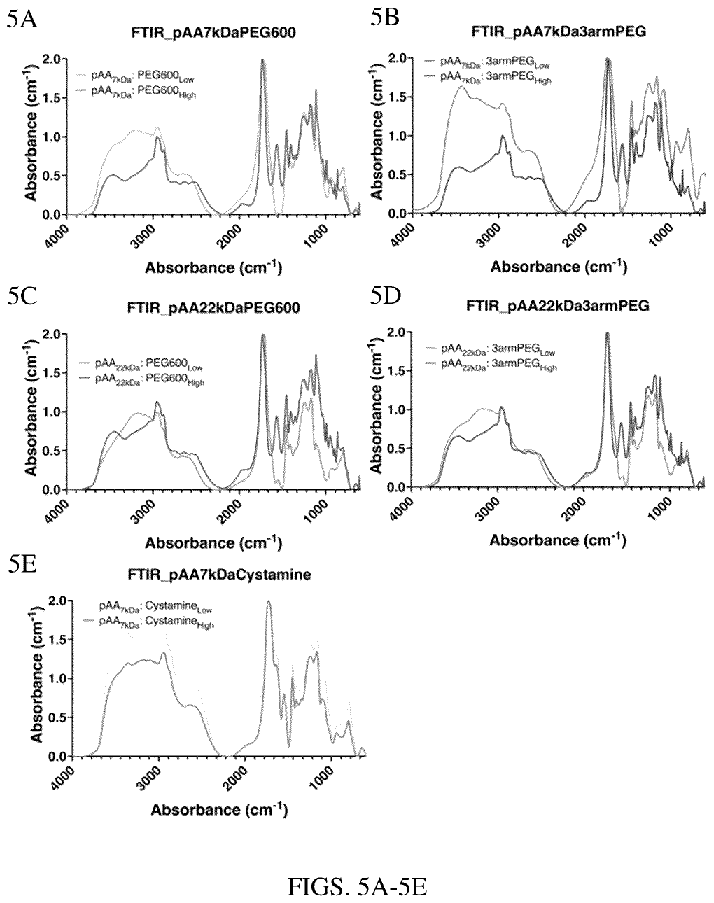

Differences in microgel crosslinking density was verified both qualitatively using FTIR. FTIR showed a decrease in the carboxylic acid peak (O—H stretch between 2500-3500 cm⁻¹) for all XLD_{Low} versus XLD_{High} microgel formulations, signifying the loss of carboxylic acid groups due to an increase in crosslinking density (FIG. 5A-5E). Additionally, for microgel formulations synthesized with either PEG600 or the 3-arm PEG, there was an increase in the ether peak strength (C—O—C stretch at 1110 cm⁻¹) when comparing XLD_{Low} versus XLD_{High} microgel formulations, which indicates a greater incorporation of the crosslinker (FIG. 5A-5D).

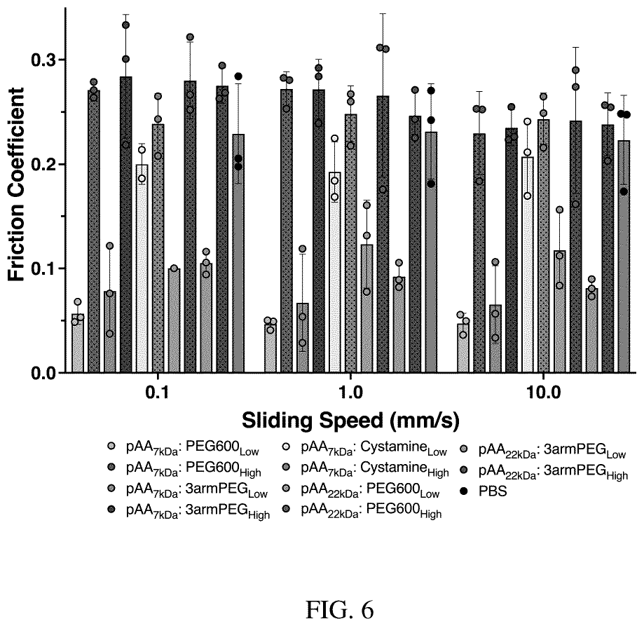

Tribological characterization of microgel suspensions was performed to determine the friction coefficients as a function of sliding speed. Across all sliding speeds, with the exception of microgels synthesized with cystamine as a linker, microgels synthesized with low crosslinking density decreased the friction coefficient compared to their respective high crosslinking density microgel counterparts (p<0.01). These results further demonstrate that microgel suspensions with low crosslinking density and various crosslinkers, regardless of pAA molecular weights, lubricate articular cartilage, while microgel batches with high crosslinking density had friction coefficients equivalent to PBS (FIG. 6). The data suggests that a wide variety of microgel formulations can be used to impart lubricity for biological tissues by controlling the crosslinking density.

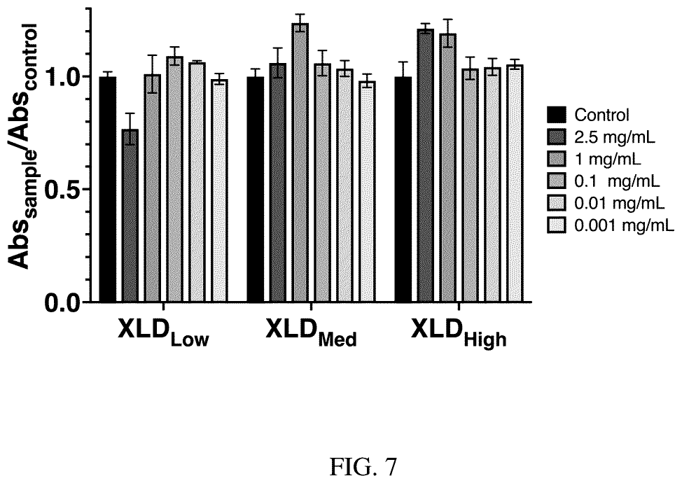

Primary chondrocytes were isolated from neonatal bovine condyles, as described above. Chondrocytes were subsequently treated with various concentrations of microgels with either low, medium, or high crosslinking density. After performing a metabolic activity assay, all treatment groups demonstrated greater than 70% metabolic activity relative to untreated control groups (FIG. 7). This data demonstrates the in vitro cytocompatibility of microgel suspensions.

While there have been shown and described what are at present considered the preferred embodiments of the invention, those skilled in the art may make various changes and modifications which remain within the scope of the invention defined by the appended claims.

What is claimed is:

1. A microgel composition comprising micron-sized particles of poly(acrylic acid) in which at least a portion of carboxylic acid groups in the poly(acrylic acid) are crosslinked as shown in the following structure: 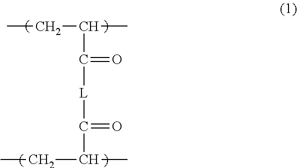
wherein:
L is a polyalkylene glycol-containing linker;
wherein the micron-sized particles have an average diameter of at least 1 micron.

2. The microgel composition of claim 1, wherein L has the following formula: —O—[CH(R)CH(R′)—O]ₓ—; wherein R and R′ are independently selected from H and CH₃; and x is an integer of 1-50.

3. The microgel composition of claim 2, wherein x is an integer of 2-20.

4. The microgel composition of claim 2, wherein x is an integer of 3-20.

5. The microgel composition of claim 2, wherein x is an integer of 4-20.

6. The microgel composition of claim 2, wherein x is an integer of 2-12.

7. The microgel composition of claim 2, wherein x is an integer of 3-12.

8. The microgel composition of claim 2, wherein x is an integer of 4-12.

9. The microgel composition of any one of claims 2-7, wherein R and R′ are both H.

10. The microgel composition of any one of claims 1-9, wherein the poly(acrylic acid) has a molecular weight of in a range of 5-60 kDa.

11. The microgel composition of any one of claims 1-9, wherein the poly(acrylic acid) has a molecular weight of in a range of 5-50 kDa.

12. The microgel composition of any one of claims 1-9, wherein the poly(acrylic acid) has a molecular weight of in a range of 5-40 kDa.

13. The microgel composition of any one of claims 1-9, wherein the poly(acrylic acid) has a molecular weight of in a range of 5-30 kDa.

14. The microgel composition of any one of claims 1-13, wherein the poly(acrylic acid) is crosslinked with the crosslinker L in a crosslinking density of at least 4%.

15. The microgel composition of any one of claims 1-13, wherein the poly(acrylic acid) is crosslinked with the crosslinker L in a crosslinking density of at least 5%.

16. The microgel composition of any one of claims 1-13, wherein the poly(acrylic acid) is crosslinked with the crosslinker L in a crosslinking density of at least 10%.

17. The microgel composition of any one of claims 1-13, wherein the poly(acrylic acid) is crosslinked with the crosslinker L in a crosslinking density of at least 20%.

18. The microgel composition of any one of claims 1-13, wherein the poly(acrylic acid) is crosslinked with the crosslinker L in a crosslinking density of at least 30%.

19. The microgel composition of any one of claims 1-18, wherein the micron-sized particles have an average diameter of 1-100 microns.

20. The microgel composition of any one of claims 1-18, wherein the micron-sized particles have an average diameter of 2-100 microns.

21. The microgel composition of any one of claims 1-18, wherein the micron-sized particles have an average diameter of 1-50 microns.

22. The microgel composition of any one of claims 1-18, wherein the micron-sized particles have an average diameter of 2-50 microns.

23. The microgel composition of any one of claims 1-22, wherein the microgel composition further comprises a therapeutic molecule suited for joint therapy or health.

24. The microgel composition of claim 23, wherein the therapeutic molecule is selected from the group consisting of non-steroidal anti-inflammatory drugs, corticosteroids, therapeutic proteins, protein inhibitors, and nucleic acids.

25. A pharmaceutical composition comprising any one of the microgel compositions of claims 1-24 in a pharmaceutically acceptable carrier.

26. A kit comprising any one of the microgel compositions of claims 1-24 and instructions for using the microgel composition.

27. A method for imparting lubricity to a biological tissue, the method comprising contacting said biological tissue with any one of the microgel compositions of claims 1-24.

28. The method of claim 27, wherein said biological tissue is selected from joints, bone, ocular tissue, nasal tissue, tendons, tendon capsule tissue, intestinal tissue, muscles, and fascia.

29. A method for treating an articular disease or condition, the method comprising administering a pharmaceutically acceptable amount of any one of the microgel compositions of claims 1-24 to a subject having an articular disease or condition.

30. The method of claim 29, wherein the articular disease or condition is arthritis.

31. The method of claim 30, wherein the arthritis is osteoarthritis.
